(19) United States
(12) Patent Application Publication
(10) Pub. No.: US 2008/0207455 A1
(43) Pub. Date: Aug. 28, 2008

(54) 5,6-Dialkyl-7-Aminoazolopyrimidines, Their Preparation and Their Use for Controlling Harmful Fungi, and Compositions Comprising These Compounds

(75) Inventors: Peter Schafer, Ottersheim (DE); Udo Hunger, Mannheim (DE); Maria Scherer, Landau (DE); Harald Kohle, Bobenheim (DE); Helmut Schiffer, Grossfischlingen (DE); Thomas Grote, Wachenheim (DE); Jochen Dietz, Mannheim (DE); Wassilios Grammenos, Ludwigshafen (DE); Jan Klaas Lohmann, Mannheim (DE); Bernd Muller, Frankenthal (DE); Joachim Rheinheimer, Ludwigshafen (DE); Frank Schieweck, Hessheim (DE); Anja Schwogler, Mannheim (DE)

Correspondence Address: HUTCHISON LAW GROUP PLLC, PO BOX 31686, RALEIGH, NC, 27612, US

(73) Assignee: BASF AKTINGESELLSCHAFT, LUDWIGSHAFEN (DE)

(21) Appl. No.: 11/885,360

(22) PCT Filed: Mar. 1, 2006
(86) PCT No.: PCT/EP06/60361

§ 371 Date: Aug. 30, 2007

(30) Foreign Application Priority Data
Mar. 1, 2005 (DE) 102005009883.5

(51) Int. Cl. A01N 43/50; C07D 487/04; A01P 3/00

(52) U.S. Cl. 504/276; 544/263

(57) ABSTRACT

5,6-Dialkyl-7-aminoazolopyrimidines of the formula I

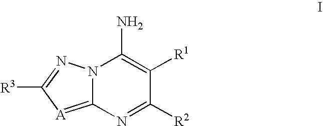

in which the substituents are as defined below:
R¹ is alkyl or alkoxyalkyl, where the aliphatic groups may be substituted as defined in the description;
R² is CHR^{x}CH₃, cyclopropyl, CH═CH₂ or CH₂CH═CH₂;
   R^{x} is hydrogen, CH₃ or CH₂CH₃ or halomethyl;
A is N or CH;
R³ is methyl,
   and, if A is CH, additionally hydrogen;
      processes and intermediates for preparing these compounds, compositions comprising them and their use for controlling phytopathogenic harmful fungi. 5,6-Dialkyl-7aminoazolopyrimidines, their preparation and their use for controlling harmful fungi, and compositions comprising these compounds

The present invention relates to 5,6-dialkyl-7-aminoazolopyrimidines of the formula I

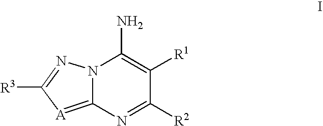

in which the substituents are as defined below:
R¹ is C₅-C₁₂-alkyl or C₅-C₁₄-alkoxyalkyl, Where the aliphatic groups may be substituted by one to three of the following groups:
   cyano, nitro, hydroxyl, C₃-C₆-cycloalkyl, C₁-C₆-alkylthio and NR^{a}R^{b};
   R^{a}, R^{b} are hydrogen or C₁-C₆-alkyl;
R² is CHR^{x}CH₃, cyclopropyl, CH═CH₂ or CH₂CH═CH₂;
   R^{x} is hydrogen, CH₃, CH₂CH₃ or halomethyl;
A is N or CH;
R³ is CH₃ and, if A is CH, additionally hydrogen.

Moreover, the invention relates to processes for preparing these compounds, to compositions comprising them and to their use for controlling phytopathogenic harmful fungi.

5,6-Dialkyl-7-aminotriazolo- and -pyrazolopyrimidines are proposed in a general manner in GB 1 148 629. Individual fungicidally active 5,6-dialkyl-7-aminotriazolo- and -pyrazolopyrimidines are known from EP-A 141 317. However, in many cases their activity is unsatisfactory. Based on this, it is an object of the present invention to provide compounds having improved activity and/or a wider activity spectrum.

We have found that this object is achieved by the compounds defined at the outset. Furthermore, we have found processes and intermediates for their preparation, compositions comprising them and methods for controlling harmful fungi using the compounds I.

The compounds of the formula I differ from those in the abovementioned publications by the specific embodiment of the substituent in the 5-position of the azolopyrimidine skeleton.

Compared to the known compounds, the compounds of the formula I are more effective against harmful fungi.

The compounds according to the invention can be obtained by different routes. Advantageously, the compounds according to the invention are obtained by reacting substituted β-keto esters of the formula II with an aminoazole of the formula III to give 7-hydroxyazolopyrimidines of the formula IV. The variables in formulae II and IV are as defined for formula I and the group R in formula II is C₁-C₄-alkyl; for practical reasons, preference is given here to methyl, ethyl or propyl.

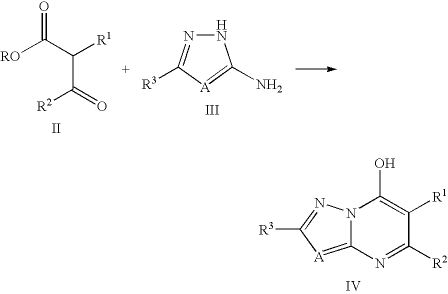

The compounds of the formula IV are novel.

The reaction of the substituted β-keto esters of the formula II with the aminoazoles of the formula III can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines and mixtures of these solvents with water. Suitable catalysts are bases as mentioned above or acids such as sulfonic acids or mineral acids. With particular preference, the reaction is carried out in the absence of a solvent or in chlorobenzene, xylene, dimethyl sulfoxide or N-methylpyrrolidone. Particularly preferred bases are tertiary amines, such as triisopropylethylamine, tributylamine, N-methylmorpholine or N-methylpiperidine. The temperatures are from 50 to 300° C., preferably from 50 to 180° C., if the reaction is carried out in solution [cf. EP-A 770 615; Adv. Het. Chem. 57 (1993), 81ff].

The bases are generally employed in catalytic amounts; however, they can also be employed in equimolar amounts, in excess or, if appropriate, as solvents.

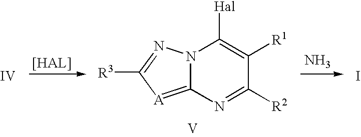

In most cases, the resulting condensates of the formula IV precipitate from the reaction solutions in pure form and, after washing with the same solvent or with water and subsequent drying, they are reacted with halogenating agents, in particular chlorinating or brominating agents, to give the compounds of the formula V in which Hal is chlorine or bromine, in particular chlorine. The reaction is preferably carried out using chlorinating agents such as phosphorus oxychloride, thionyl chloride or sulfuryl chloride at from 50° C. to 150° C., preferably in excess phosphorus oxytrichloride at reflux temperature. After evaporation of excess phosphorus oxytrichloride, the residue is treated with ice-water, if appropriate with addition of a water-immiscible solvent. In most cases, the chlorinated product isolated from the dried organic phase, if appropriate after evaporation of the inert solvent, is very pure and is subsequently reacted with ammonia in inert solvents at from 100° C. to 200° C. to give the 7-amino-azolo[1,5-a]pyrimidines. The reaction is preferably carried out using a 1- to 10-molar excess of ammonia, under a pressure of from 1 to 100 bar.

The novel 7-aminoazolo[1,5-a]pyrimidines are, if appropriate after evaporation of the solvent, isolated as crystalline compounds, by digestion in water.

The β-keto esters of the formula II can be prepared as described in Organic Synthesis Coll. Vol. 1, p. 248, and/or they are commercially available.

The intermediates of the formula V are novel.

Alternatively, the novel compounds of the formula I can be obtained by reacting substituted acyl cyanides of the formula VI in which R¹ and R² are as defined above with an aminoazole of the formula III.

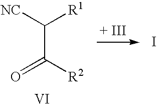

The reaction can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as those mentioned above, and mixtures of these solvents with water. The reaction temperatures are from 50 to 300° C., preferably from 50 to 150° C., if the reaction is carried out in solution.

The novel 7-aminoazolo[1,5-a]pyrimidines of the formula I are, if appropriate after evaporation of the solvent or dilution with water, isolated as crystalline compounds.

Some of the substituted alkyl cyanides of the formula VI required for preparing the 7-aminoazolo[1,5-a]pyrimidines are known, or they can be prepared by known methods from alkyl cyanides and carboxylic acid esters using strong bases, for example alkali metal hydrides, alkali metal alkoxides, alkali metal amides or metal alkyls (cf.: J. Amer. Chem. Soc. 73, (1951), p. 3766).

If individual compounds I can not be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

In the definitions of symbols given in the formulae above, collective terms were used which are generally representative of the following substituents:

halogen: fluorine, chlorine, bromine and iodine;

alkyl: saturated straight-chain or mono- or dibranched hydrocarbon radicals having 1 to 4 or 5 to 12 carbon atoms, for example C₁-C₆-alkyl such as methyl, ethyl, propyl, 1-methylethyl, butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

halomethyl: a methyl group in which some or all of the hydrogen atoms may be replaced by halogen atoms as mentioned above; in particular chloromethyl, bromomethyl, dichloromethyl, trichloromethyl, fluoromethyl, difluoromethyl, trifluoromethyl, chlorofluoromethyl, dichlorofluoromethyl, chlorodifluoromethyl;

cycloalkyl: mono- or bicyclic saturated hydrocarbon groups having 3 to 6 carbon ring members, such as cyclopropyl, cyclobutyl, cyclopentyl and cyclohexyl;

alkoxyalkyl: a saturated straight-chain or mono-, di- or tribranched hydrocarbon chain which is interrupted by an oxygen atom, for example C₅-C₁₂-alkoxyalkyl: a hydrocarbon chain as described above having 5 to 12 carbon atoms which may be interrupted by an oxygen atom in any position, such as propoxyethyl, butoxyethyl, pentoxyethyl, hexyloxyethyl, heptyloxyethyl, octyloxyethyl, nonyloxyethyl, 3-(3-ethylhexyloxy)ethyl, 3-(2,4,4-trimethylpentyloxy)ethyl, 3-(1-ethyl-3-methylbutoxy)ethyl, ethoxypropyl, propoxypropyl, butoxypropyl, pentoxypropyl, hexyloxypropyl, heptyloxypropyl, octyloxypropyl, nonyloxypropyl, 3-(3-ethylhexyloxy)propyl, 3-(2,4,4-trimethylpentyloxy)propyl, 3-(1-ethyl-3-methylbutoxy)propyl, ethoxybutyl, propoxybutyl, butoxybutyl, pentoxybutyl, hexyloxybutyl, heptyloxybutyl, octyloxybutyl, nonyloxybutyl, 3-(3-ethylhexyloxy)butyl, 3-(2,4,4-trimethylpentyloxy)butyl, 3-(1-ethyl-3-methylbutoxy)butyl, methoxypentyl, ethoxypentyl, propoxypentyl, butoxypentyl, pentoxypentyl, hexyloxypentyl, heptyloxypentyl, 3-(3-methylhexyloxy)pentyl, 3-(2,4-dimethylpentyloxy)pentyl, 3-(1-ethyl-3-methylbutoxy)pentyl.

The scope of the present invention includes the (R)- and (S)-isomers and the racemates of compounds of the formula I having chiral centers.

With a view to the intended use of the azolopyrimidines of the formula I, particular preference is given to the following meanings of the substituents, in each case on their own or in combination:

Preference is given to compounds I in which the group R¹ has at most 12 carbon atoms.

The alkyl groups in R¹ in formula I are preferably straight-chain or mono-, di-, tri- or multibranched alkyl groups, in particular a straight-chain alkyl group.

The alkoxyalkyl groups in R¹ in formula I are preferably C₃-C₁₂-alkoxyethyl or, in particular, C₂-C¹¹-alkoxypropyl groups.

In addition, preference is given to compounds of the formula I which, in R¹, are branched at the α carbon atom. They are described by formula Ia:

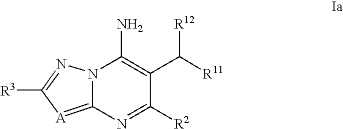

in which R¹¹ is C₃-C₁₀-alkyl or C₅-C₁₀-alkoxyalkyl and R¹² is C₁-C₄-alkyl, in particular methyl, where R¹¹ and R¹² together have at most 12 carbon atoms and are unsubstituted or may be substituted like R¹ in formula I.

If R¹ is an alkyl group substituted by cyano, the cyano group is preferably located at the terminal carbon atom.

Preference is given to compounds I in which R¹ is a straight-chain or mono-, di-, tri- or multibranched C₅-C₁₂-alkyl group which does not carry any further substituents.

In one embodiment of the compounds I according to the invention, R¹ is C₅-C₁₂-alkyl or C₁-C₁₁-alkoxy-C₁-C₁₁-alkyl, where the total number of carbon atoms preferably has a value of from 5 to 12. Here, particular preference is given to C₂-C₉-alkoxy-propyl groups.

Particular preference is given to compounds I in which R¹ is n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl.

In addition, preference is given to compounds of the formula I in which R¹ is n-heptyl, 1-methylhexyl, n-octyl, 1-methylheptyl, n-nonyl, 1-methyloctyl, 3,5,5-trimethylhexyl, n-decyl, 1-methyinonyl, n-undecyl, 1-methyldecyl, n-dodecyl and 1-methylundecyl.

In one preferred embodiment of the compounds I according to the invention, R² is ethyl.

In a further embodiment of the compounds I according to the invention, R³ is methyl.

In a further embodiment of the compounds I according to the invention, R³ is hydrogen.

One embodiment of the compounds according to the invention relates to compounds I in which A is CH. These compounds correspond to formula I.1:

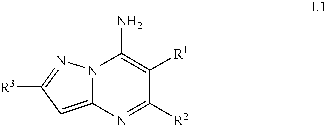

Another embodiment of the compounds according to the invention relates to compounds I in which A is N. These compounds correspond to formula I.2:

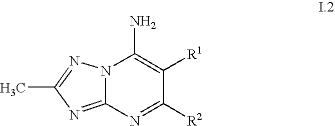

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

TABLE 1

Compounds of the formula I.1 in which R¹ for each compound
corresponds to one row of Table A and R² is ethyl and R³ is
hydrogen

TABLE 2

Compounds of the formula I.1 in which R¹ for each compound
corresponds to one row of Table A and R² is isopropyl and
R³ is hydrogen

TABLE 3

Compounds of the formula I.1 in which R¹ for each compound
corresponds to one row of Table A and R² is 1-methylpropyl
and R³ is hydrogen

TABLE 4

Compounds of the formula I.1 in which R¹ for each compound
corresponds to one row of Table A and R² is ethenyl and R³
is hydrogen

TABLE 5

Compounds of the formula I.1 in which R¹ for each compound
corresponds to one row of Table A and R² is allyl and R³ is
hydrogen

TABLE 6

Compounds of the formula I.1 in which R¹ for each compound
corresponds to one row of Table A and R² is cyclopropyl
and R³ is hydrogen

TABLE 7

Compounds of the formula I.1 in which R¹ for each compound
corresponds to one row of Table A and R² is ethyl and R³ is
methyl

TABLE 8

Compounds of the formula I.1 in which R¹ for each compound
corresponds to one row of Table A and R² is isopropyl and
R³ is methyl

TABLE 9

Compounds of the formula I.1 in which R¹ for each compound
corresponds to one row of Table A and R² is 1-methylpropyl
and R³ is methyl

TABLE 10

Compounds of the formula I.1 in which R¹ for each compound
corresponds to one row of Table A and R² is ethenyl and R³
is methyl

TABLE 11

Compounds of the formula I.1 in which R¹ for each compound
corresponds to one row of Table A and R² is allyl and R³ is
methyl

TABLE 12

Compounds of the formula I.1 in which R¹ for each compound
corresponds to one row of Table A and R² is cyclopropyl and
R³ is methyl

TABLE 13

Compounds of the formula I.2 in which R¹ for each compound
corresponds to one row of Table A and R² is ethyl

TABLE 14

Compounds of the formula I.2 in which R¹ for each compound
corresponds to one row of Table A and R² is isopropyl

TABLE 15

Compounds of the formula I.2 in which R¹ for each compound
corresponds to one row of Table A and R² is 1-methylpropyl

TABLE 16

Compounds of the formula I.2 in which R¹ for each compound
corresponds to one row of Table A and R² is ethenyl

TABLE 17

Compounds of the formula I.2 in which R¹ for each compound
corresponds to one row of Table A and R² is allyl

TABLE 18

Compounds of the formula I.2 in which R¹ for each compound
corresponds to one row of Table A and R² is cyclopropyl.

TABLE A

| No. | R¹ |
|---|---|
| A-1 | CH₂CH₂CH₂CH₂CH₃ |
| A-2 | CH(CH₃)CH₂CH₂CH₃ |
| A-3 | CH₂CH(CH₃)CH₂CH₃ |
| A-4 | CH₂CH₂CH(CH₃)CH₃ |
| A-5 | CH₂CH₂CH(CH₃)₂ |
| A-6 | CH(CH₃)CH(CH₃)CH₃ |
| A-7 | CH(CH₃)CH(CH₃)₂ |
| A-8 | CH₂C(CH₃)₃ |
| A-9 | CH₂CH₂CH₂CH₂CH₂CH₃ |
| A-10 | CH(CH₃)CH₂CH₂CH₂CH₃ |
| A-11 | CH₂CH(CH₃)CH₂CH₂CH₃ |
| A-12 | CH₂CH₂CH(CH₃)CH₂CH₃ |
| A-13 | CH₂CH₂CH(CH₃)₂CH₂ |
| A-14 | CH₂CH₂CH₂CH(CH₃)₂ |
| A-15 | CH(CH₃)CH(CH₃)CH₂CH₃ |
| A-16 | CH(CH₃)CH₂CH(CH₃)₂ |
| A-17 | CH₂CH₂C(CH₃)₃ |
| A-18 | CH(CH₃)CH₂CH(CH₃)CH₃ |
| A-19 | CH₂CH₂CH₂CH₂CH₂CH₂CH₃ |
| A-20 | CH(CH₃)CH₂CH₂CH₂CH₂CH₃ |
| A-21 | CH₂CH(CH₃)CH₂CH₂CH₂CH₃ |
| A-22 | CH₂CH₂CH(CH₃)CH₂CH₂CH₃ |
| A-23 | CH₂CH₂CH₂CH(CH₃)CH₂CH₃ |
| A-24 | CH₂CH₂CH₂CH₂CH(CH₃)CH₃ |
| A-25 | CH₂CH₂CH₂CH₂CH(CH₃)₂ |
| A-26 | CH(CH₃)CH(CH₃)CH₂CH₂CH₃ |
| A-27 | CH₂CH(CH₃)CH(CH₃)CH₂CH₃ |
| A-28 | CH₂CH₂CH₂C(CH₃)₃ |
| A-29 | CH(CH₃)CH₂CH(CH₃)CH₂CH₃ |
| A-30 | CH₂CH(CH₃)CH(CH₃)CH₂CH₃ |
| A-31 | CH(CH₃)CH₂CH₂CH(CH₃)CH₃ |
| A-32 | CH₂CH₂CH₂CH₂CH₂CH₂CH₂CH₃ |
| A-33 | CH(CH₃)CH₂CH₂CH₂CH₂CH₂CH₃ |
| A-34 | CH₂CH(CH₃)CH₂CH₂CH₂CH₂CH₃ |
| A-35 | CH₂CH₂CH(CH₃)CH₂CH₂CH₂CH₃ |
| A-36 | CH₂CH₂CH₂CH(CH₃)CH₂CH₂CH₃ |
| A-37 | CH₂CH₂CH₂CH₂CH(CH₃)CH₂CH₃ |
| A-38 | CH₂CH₂CH₂CH₂CH₂CH(CH₃)₂ |
| A-39 | CH₂CH₂CH₂CH₂C(CH₃)₃ |
| A-40 | CH(CH₃)CH(CH₃)CH₂CH₂CH₂CH₃ |
| A-41 | CH₂CH(CH₃)CH(CH₃)CH₂CH₂CH₃ |
| A-42 | CH₂CH₂CH₂C(CH₃)₂CH₂CH₃ |
| A-43 | CH(CH₃)CH₂CH(CH₃)CH₂CH₂CH₃ |
| A-44 | CH₂CH(CH₃)CH(CH₃)CH₂CH₂CH₃ |
| A-45 | CH(CH₃)CH₂CH₂CH(CH₃)CH₂CH₃ |
| A-46 | CH(CH₃)CH₂CH₂CH₂CH(CH₃)₂ |
| A-47 | CH₂CH₂CH(CH₃)CH₂C(CH₃)₃ |
| A-48 | CH₂CH₂CH₂CH₂CH₂CH₂CH₂CH₂CH₃ |
| A-49 | CH(CH₃)CH₂CH₂CH₂CH₂CH₂CH₂CH₃ |
| A-50 | CH₂CH(CH₃)CH₂CH₂CH₂CH₂CH₂CH₃ |
| A-51 | CH₂CH₂CH(CH₃)CH₂CH₂CH₂CH₂CH₃ |
| A-52 | CH₂CH₂CH₂CH(CH₃)CH₂CH₂CH₂CH₃ |
| A-53 | CH₂CH₂CH₂CH₂CH(CH₃)CH₂CH₂CH₃ |
| A-54 | CH₂CH₂CH₂CH₂CH₂CH₂C(CH₃)₃ |
| A-55 | CH(CH₃)CH(CH₃)CH₂CH₂CH₂CH₂CH₃ |
| A-56 | CH₂CH(CH₃)CH(CH₃)CH₂CH₂CH₂CH₃ |
| A-57 | CH₂CH₂CH₂C(CH₃)₂CH₂CH₂CH₃ |
| A-58 | CH(CH₃)CH₂CH(CH₃)CH₂CH₂CH₂CH₃ |
| A-59 | CH₂CH(CH₃)CH(CH₃)CH₂CH₂CH₂CH₃ |
| A-60 | CH(CH₃)CH₂CH₂CH(CH₃)CH₂CH₂CH₃ |
| A-61 | CH(CH₃)CH₂CH₂CH₂C(CH₃)₃ |
| A-62 | CH₂CH(CH₃)CH₂CH₂CH(CH₃)₃ |
| A-63 | CH(CH₃)CH₂CH₂CH₂CH₂CH(CH₃)₂ |
| A-64 | CH₂CH(CH₃)CH₂CH₂CH₂CH(CH₃)₂ |
| A-65 | CH₂CH₂CH₂CH₂CH₂CH₂CH₂CH₂CH₂CH₃ |
| A-66 | CH(CH₃)CH₂CH₂CH₂CH₂CH₂CH₂CH₂CH₃ |
| A-67 | CH₂CH(CH₃)CH₂CH₂CH₂CH₂CH₂CH₂CH₃ |
| A-68 | CH₂CH₂CH(CH₃)CH₂CH₂CH₂CH₂CH₂CH₃ |
| A-69 | CH₂CH₂CH(CH₃)CH₂CH₂CH₂CH₂CH₂ |
| A-70 | CH₂CH₂CH₂CH(CH₃)CH₂CH₂CH₂CH₃ |
| A-71 | CH₂CH₂CH₂CH₂CH₂CH₂C(CH₃)₃ |
| A-72 | CH(CH₃)CH(CH₃)CH₂CH₂CH₂CH₂CH₂CH₃ |
| A-73 | CH₂CH(CH₃)CH(CH₃)CH₂CH₂CH₂CH₂CH₃ |
| A-74 | CH₂CH₂CH₂C(CH₃)₂CH₂CH₂CH₂CH₃ |
| A-75 | CH(CH₃)CH₂CH(CH₃)CH₂CH₂CH₂CH₂CH₃ |
| A-76 | CH₂CH(CH₃)CH(CH₃)CH₂CH₂CH₂CH₂CH₃ |
| A-77 | CH(CH₃)CH₂CH₂CH(CH₃)CH₂CH₂CH₂CH₃ |
| A-78 | CH(CH₃)CH₂CH₂CH₂CH(CH₃)CH₂CH₂CH₃ |
| A-79 | CH(CH₃)CH₂CH₂CH₂CH₂CH(CH₃)CH₂CH₃ |
| A-80 | CH(CH₃)CH₂CH₂CH₂CH₂CH₂CH(CH₃)₂ |
| A-81 | CH(CH₃)CH₂CH₂CH₂CH₂CH₂C(CH₃)CH₃ |
| A-82 | CH₂CH(CH₃)CH₂CH₂CH₂CH₂CH(CH₃)CH₃ |
| A-83 | CH(CH₃)CH₂CH₂CH₂CH₂C(CH₃)₃ |
| A-84 | CH₂CH(CH₃)CH₂CH₂CH₂C(CH₃)₃ |
| A-85 | CH₂CH₂CH₂CH₂CH₂CH₂CH₂CH₂CH₂CH₂CH₃ |
| A-86 | CH(CH₃)CH₂CH₂CH₂CH₂CH₂CH₂CH₂CH₂CH₃ |
| A-87 | CH₂CH(CH₃)CH₂CH₂CH₂CH₂CH₂CH₂CH₂CH₃ |
| A-88 | CH₂CH₂CH(CH₃)CH₂CH₂CH₂CH₂CH₂CH₂CH₃ |
| A-89 | CH₂CH₂CH₂CH(CH₃)CH₂CH₂CH₂CH₂CH₂CH₃ |
| A-90 | CH₂CH₂CH₂CH₂CH(CH₃)CH₂CH₂CH₂CH₂CH₃ |
| A-91 | CH₂CH₂CH₂CH₂CH₂CH₂CH₂C(CH₃)₃ |
| A-92 | CH(CH₃)CH(CH₃)CH₂CH₂CH₂CH₂CH₂CH₂CH₃ |
| A-93 | CH₂CH(CH₃)CH(CH₃)CH₂CH₂CH₂CH₂CH₂CH₃ |
| A-94 | CH₂CH₂CH₂C(CH₃)₂CH₂CH₂CH₂CH₂CH₃ |
| A-95 | CH(CH₃)CH₂CH(CH₃)CH₂CH₂CH₂CH₂CH₂CH₃ |
| A-96 | CH₂CH(CH₃)CH(CH₃)CH₂CH₂CH₂CH₂CH₂CH₃ |
| A-97 | CH(CH₃)CH₂CH₂CH(CH₃)CH₂CH₂CH₂CH₂CH₃ |
| A-98 | CH(CH₃)CH₂CH₂CH₂CH(CH₃)CH₂CH₂CH₂CH₃ |
| A-99 | CH(CH₃)CH₂CH₂CH₂CH₂CH(CH₃)CH₂CH₂CH₃ |
| A-100 | CH(CH₃)CH₂CH₂CH₂CH₂CH₂CH(CH₃)CH₂CH₃ |
| A-101 | CH(CH₃)CH₂CH₂CH₂CH₂CH₂CH₂CH(CH₃)₂ |
| A-102 | CH₂CH(CH₃)CH₂CH₂CH₂CH₂CH(CH₃)CH₂CH₃ |
| A-103 | CH₂CH(CH₃)CH₂CH₂CH₂CH₂CH(CH₃)CH₂CH₃ |
| A-104 | CH₂CH₂CH(CH₃)CH₂CH₂CH₂CH₂CH(CH₃)₂ |
| A-105 | CH₂CH(CH₃)CH₂CH₂CH₂CH₂C(CH₃)₃ |
| A-106 | CH₂CH₂CH₂CH₂CH₂CH₂CH₂CH₂CH₂CH₂CH₂CH₃ |
| A-107 | CH(CH₃)CH₂CH₂ CH₂CH₂CH₂CH₂CH₂CH₂CH₂CH₃ |
| A-108 | CH₂CH(CH₃)CH₂CH₂CH₂CH₂CH₂CH₂CH₂CH₂CH₃ |
| A-109 | CH₂CH₂CH(CH₃)CH₂CH₂CH₂CH₂CH₂CH₂CH₂CH₃ |
| A-110 | CH₂ CH₂CH₂CH(CH₃)CH₂CH₂CH₂CH₂CH₂CH₂CH₂ |
| A-111 | CH₂CH₂CH₂CH₂CH(CH₃)CH₂CH₂CH₂CH₂CH₂CH₃ |
| A-112 | CH₂CH₂CH₂CH₂CH₂CH(CH₃)CH₂CH₂CH₂CH₂CH₃ |
| A-113 | CH₂CH₂CH₂CH₂CH₂CH₂CH(CH₃)CH₂CH₂CH₂CH₃ |
| A-114 | CH₂CH₂CH₂CH₂CH₂CH₂CH₂CH(CH₃)CH₂CH₂CH₃ |
| A-115 | CH₂CH₂CH₂CH₂CH₂CH₂CH₂CH₂CH(CH₃)CH₂CH₃ |
| A-116 | CH₂CH₂CH₂CH₂CH₂CH₂CH₂CH₂CH₂CH(CH₃)₂ |
| A-117 | CH(CH₃)CH(CH₃)CH₂CH₂CH₂CH₂CH₂CH₂CH₂CH₃ |
| A-118 | CH₂CH(CH₃)CH(CH₃)CH₂CH₂CH₂CH₂CH₂CH₂CH₃ |
| A-119 | CH₂CH₂CH₂C(CH₃)₂CH₂CH₂CH₂CH₂CH₂CH₃ |
| A-120 | CH₂CH₂CH₂CH₂CH(CH₃)CH₂CH₂CH₂CH₂CH₂CH₃ |
| A-121 | CH(CH₃)CH₂CH(CH₃)CH₂CH₂CH₂CH₂ CH₂CH₂CH₃ |
| A-122 | CH(CH₃)CH₂CH₂CH(CH₃)CH₂CH₂CH₂ CH₂CH₂CH₃ |
| A-123 | CH(CH₃)CH₂CH₂CH₂CH(CH₃)CH₂CH₂CH₂CH₂CH₃ |
| A-124 | CH(CH₃)CH₂CH₂CH₂CH₂CH(CH₃)CH₂CH₂CH₂CH₃ |
| A-125 | CH(CH₃)CH₂CH₂CH₂CH₂OH₂CH(CH₃)CH₂CH₂CH₃ |
| A-126 | CH(CH₃)CH₂CH₂CH₂CH₂CH₂CH₂CH(CH₃)CH₂CH₃ |
| A-127 | CH₂CH(CH₃)CH₂CH₂CH₂CH₂CH(CH₃)CH₂CH₂CH₃ |
| A-128 | CH₂CH₂CH(CH₃)CH₂CH₂CH₂CH₂CH(CH₃)CH₂CH₃ |
| A-129 | CH₂CH₂CH₂CH(CH₃)CH₂CH₂CH₂CH(CH₃)CH₂CH₃ |
| A-130 | CH₂CH(CH₃)CH₂CH₂CH₂CH₂CH₂C(CH₃)₃ |
| A-131 | CH₂CH₂CH₂—O—CH₃ |
| A-132 | CH₂CH₂CH₂—O—CH₂CH₃ |
| A-133 | CH₂CH₂CH₂—O—CH₂CH₂CH₃ |
| A-134 | CH₂CH₂CH₂—O—CH₂CH₂CH₂CH₃ |
| A-135 | CH₂CH₂CH₂—O—CH₂CH₂CH₂CH₂CH₃ |
| A-136 | CH₂CH₂CH₂—O—CH₂CH₂CH₂CH₂CH₂CH₃ |
| A-137 | CH₂CH₂CH₂—O—CH₂CH₂CH₂CH₂CH₂CH₂CH₃ |
| A-138 | CH₂CH₂CH₂—O—CH₂CH₂CH₂CH₂CH₂CH₂CH₂CH₃ |
| A-139 | CH₂CH₂CH₂—O—CH₂CH₂CH₂CH₂CH₂CH₂CH₂CH₂CH₃ |
| A-140 | CH₂CH₂CH₂—O—CH(CH₃)₂ |
| A-141 | CH₂CH₂CH₂—O—C(CH₃)₃ |
| A-142 | CH₂CH₂CH₂—O—CH₂C(CH₃)₃ |
| A-143 | CH₂CH₂CH₂—O—CH(CH₃)CH₂C(CH₃)₃ |
| A-144 | CH₂CH₂CH₂—O—CH(CH₂CH₃)CH₂C(CH₃)₃ |
| A-145 | CH₂CH₂CH₂—O—CH₂CH(CH₃)CH₂CH(CH₃)₂ |
| A-146 | CH₂CH₂CH₂—O—CH₂CH(CH₂CH₃)CH₂CH₂CH₃ |
| A-147 | CH₂CH₂CH₂—O—CH₂CH₂CH(CH₃)CH₂CH(CH₃)₂ |
| A-148 | CH₂CH₂CH₂—O—CH₂CH₂CH(CH₃)CH₂C(CH₃)₃ |
| A-149 | CH₂CH₂CH₂—O—CH₂CH₂CH(CH₃)CH₂CH₂CH(CH₃)₂ |
| A-150 | CH₂CH₂CH₂—O—CH₂CH₂CH(CH₃)CH₂CH₂CH₂CH(CH₃)₂ |
| A-151 | CH₂CH₂CH₂CH₂—O—CH₃ |
| A-152 | CH₂CH₂CH₂CH₂—O—CH₂CH₃ |
| A-153 | CH₂CH₂CH₂CH₂—O—CH₂CH₂CH₃ |
| A-154 | CH₂CH₂CH₂CH₂—O—CH₂CH₂CH₂CH₃ |
| A-155 | CH₂CH₂CH₂CH₂—O—CH₂CH₂CH₂CH₂CH₃ |
| A-156 | CH₂CH₂CH₂CH₂—O—CH₂CH₂CH₂CH₂CH₂CH₃ |
| A-157 | CH₂CH₂CH₂CH₂—O—CH₂CH₂CH₂CH₂CH₂CH₂CH₃ |
| A-158 | CH₂CH₂CH₂CH₂—O—CH₂CH₂CH₂CH₂CH₂CH₂CH₂CH₃ |
| A-159 | CH₂CH₂CH₂CH₂—O—CH(CH₃)₂ |
| A-160 | CH₂CH₂CH₂CH₂—O—C(CH₃)₃ |
| A-161 | CH₂CH₂CH₂CH₂—O—CH₂C(CH₃)₃ |
| A-162 | CH₂CH₂CH₂CH₂—O—CH(CH₃)CH₂C(CH₃)₃ |
| A-163 | CH₂CH₂CH₂CH₂—O—CH(CH₂CH₃)CH₂C(CH₃)₃ |
| A-164 | CH₂CH₂CH₂CH₂—O—CH₂CH(CH₃)CH₂CH(CH₃)₂ |
| A-165 | CH₂CH₂CH₂CH₂—O—CH₂CH(CH₂CH₃)CH₂CH₂CH₃ |
| A-166 | CH₂CH₂CH₂CH₂—O—CH₂CH₂CH(CH₃)CH₂CH(CH₃)₂ |
| A-167 | CH₂CH₂CH₂CH₂—O—CH₂CH₂CH(CH₃)CH₂C(CH₃)₃ |
| A-168 | CH₂CH₂CH₂CH₂—O—CH₂CH₂CH(CH₃)CH₂CH₂CH(CH₃)₂ |
| A-169 | CH₂CH₂CH₂CH₂—O— |
| | CH₂CH₂CH(CH₃)CH₂CH₂CH₂CH(CH₃)₂ |
| A-170 | CH₂CH₂CH₂CH₂CH₂—O—CH₃ |
| A-171 | CH₂CH₂CH₂CH₂CH₂—O—CH₂CH₃ |
| A-172 | CH₂CH₂CH₂CH₂CH₂—O—CH₂CH₂CH₃ |
| A-173 | CH₂CH₂CH₂CH₂CH₂—O—CH₂CH₂CH₂CH₃ |
| A-174 | CH₂CH₂CH₂CH₂CH₂—O—CH₂CH₂CH₂CH₂CH₃ |
| A-175 | CH₂CH₂CH₂CH₂CH₂—O—CH₂CH₂CH₂CH₂CH₂CH₃ |
| A-176 | CH₂CH₂CH₂CH₂CH₂—O—CH₂CH₂CH₂CH₂CH₂CH₂CH₃ |
| A-177 | CH₂CH₂CH₂CH₂CH₂—O—CH₂CH₂CH₂CH₂CH₂CH₂CH₂CH₃ |
| A-178 | CH₂CH₂CH₂CH₂CH₂—O—CH(CH₃)₂ |
| A-179 | CH₂CH₂CH₂CH₂CH₂—O—C(CH₃)₃ |
| A-180 | CH₂CH₂CH₂CH₂CH₂—O—CH₂C(CH₃)₃ |
| A-181 | CH₂CH₂CH₂CH₂CH₂—O—CH(CH₃)CH₂C(CH₃)₃ |
| A-182 | CH₂CH₂CH₂CH₂CH₂—O—CH(CH₂CH₃)CH₂C(CH₃)₃ |
| A-183 | CH₂CH₂CH₂CH₂CH₂—O—CH₂CH(CH₃)CH₂CH(CH₃)₂ |
| A-184 | CH₂CH₂CH₂CH₂CH₂—O—CH₂CH(CH₂CH₃)CH₂CH₂CH₃ |
| A-185 | CH₂CH₂CH₂CH₂CH₂—O— |
| | CH₂CH₂CH(CH₃)CH₂CH₂CH(CH₃)₂ |
| A-186 | CH₂CH₂CH₂CH₂CH₂—O—CH₂CH₂CH(CH₃)CH₂CH(CH₃)₂ |
| A-187 | CH₂CH₂CH₂CH₂CH₂—O—CH₂CH₂CH(CH₃)CH₂C(CH₃)₃ |

The compounds I are suitable as fungicides. They are distinguished by an outstanding effectiveness against a broad spectrum of phytopathogenic fungi from the classes of the Ascomycetes, Deuteromycetes, Oomycetes and Basidiomycetes, especially from the class of the Oomycetes. Some are systemically effective and they can be used in plant protection as foliar and soil fungicides.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Altemaria species on vegetables, rapeseed, sugar beet and fruit and rice (for example A. solani or A. alternata on potato and other plants),
Aphanomyces species on sugar beet and vegetables,
Bipolaris and Drechslera species on corn, cereals, rice and lawns (for example D. teres on barley, D. tritci-repentis on wheat),
Blumeria graminis (powdery mildew) on cereals,
Botrytis cinerea (gray mold) on strawberries, vegetables, flowers and grapevines,
Bremia lactucae on lettuce,
Cercospora species on corn, soybeans, rice and sugar beet (for example C. beticula on sugar beet),
Cochliobolus species on corn, cereals, rice (for example Cochliobolus sativus on cereals, Cochliobolus miyabeanus on rice),
Colletotricum species on soybeans, cotton and other plants (for example C. acutatum on various plants),
Exserohilum species on corn,
Erysiphe cichoracearum and Sphaerotheca fuliginea on cucurbits,
Fusarium and Verticillium species (for example V. dahliae) on various plants (for example F. graminearum on wheat),
Gaeumanomyces graminis on cereals,
Gibberella species on cereals and rice (for example Gibberella fujikuroi on rice),
Grainstaining complex on rice,
Helminthosporium species (for example H. graminicola) on corn and rice,
Michrodochium nivale on cereals,
Mycosphaerella species on cereals, bananas and peanuts (M. graminicola on wheat, M. fijiesis on bananas),
Phakopsara pachyrhizi and Phakopsara meibomiae on soybeans,
Phomopsis species on soybeans, sunflowers and grapevines (P. viticola on grapevines, P. helianthii on sunflowers),
Phytophthora infestans on potatoes and tomatoes,
Plasmopara viticola on grapevines,
Podosphaera leucotricha on apples,
Pseudocercosporella herpotrichoides on cereals,
Pseudoperonospora species on hops and cucurbits (for example P. cubenis on cucumbers),
Puccinia species on cereals, corn and asparagus (P. triticina and P. striformis on wheat, P. asparagi on asparagus),
Pyrenophora species on cereals,
Pyricularia oryzae, Corticium sasakii, Sarocladium oryzae, S. attenuatum, Entyloma oryzae on rice,
Pyricularia grisea on lawns and cereals,
Pythium spp. on lawns, rice, corn, cotton, rapeseed, sunflowers, sugar beet, vegetables and other plants,
Rhizoctonia-species (for example R. solani) on cotton, rice, potatoes, lawns, corn, rapeseed, potatoes, sugar beet, vegetables and other plants,
Sclerotinia species (for example S. sclerotiorum) on rapeseed, sunflowers and other plants,
Septoria tritici and Stagonospora nodorum on wheat,
Erysiphe (syn. Uncinula necator) on grapevines,
Setospaeria species on corn and lawns,
Sphacelotheca reilinia on corn,
Thievaliopsis species on soybeans and cotton,
Tilletia species on cereals,
Ustilago species on cereals, corn and sugar beet and
Venturia species (scab) on apples and pears (for example V. inaequalis on apples).

They are particularly suitable for controlling harmful fungi from the class of the Oomycetes, such as Peronospora species, Phytophthora species, Plasmopara viticola and Pseudoperonospora species.

The compounds I are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise from 0.1 to 95%, preferably from 0.5 to 90%, by weight of active compound.

When employed in plant protection, the amounts applied are, depending on the kind of effect desired, from 0.01 to 2.0 kg of active compound per ha.

In seed treatment, amounts of active compound of 1 to 1000 g/100 kg, preferably 5 to 100 g/100 kg, of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds of the formula I can be present in various crystal modifications which may differ in their biological activity. They also form part of the project matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutyinaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ethers, ethoxylated isooctylphenol, octylphenol, nonylphenol, alkylphenol polyglycol ethers, tributylphenyl polyglycol ethers, tristearylphenyl polyglycol ethers, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved in 90 parts by weight of water or in a water-soluble solvent. As an alternative, wetting agents or other auxiliaries are added. The active compound dissolves upon dilution with water. In this way, a formulation having a content of 10% by weight of active compound is obtained.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight.

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetting agents and silica gel. Dilution with water gives a stable dispersion or solution of the active compound. The active compound content of the formulation is 75% by weight.

### H Gel Formulations

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

### I Dustable Powders (DP, DS)

5 parts by weight of the active compounds are ground finely and mixed intimately with 95 parts by weight of finely divided kaolin. This gives a dustable product having an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules to be applied undiluted having an active compound content of 0.5% by weight.

K ULV solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the use form as fungicides, also be present together with other active compounds, e.g. with herbicides, insecticides, growth regulators, fungicides or else with fertilizers. Mixing the compounds I or the compositions comprising them with one or more other active compounds, in particular fungicides, it is in many cases possible, for example to broaden the activity spectrum or to prevent the development of resistance. In many cases, synergistic effects are obtained.

The following list of fungicides, in conjunction with which the compounds according to the invention can be used, is intended to illustrate the possible combinations but does not limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

The procedures described in the following synthesis examples were used to prepare further compounds I by appropriate modification of the starting materials. The compounds thus obtained are listed in the table below, together with physical data.

### Example 1

### Preparation of 5-ethyl-2-methyl-6-(3,5,5-trimethylhexyl)-[1,2,4]triazolo[1,5-a]pyrimidin-7-ylamine [I-1]

### Example 1a

### Methyl 5,7,7-trimethyl-2-propionyloct-2-enoate

At approximately 0° C. 13.0 g of methyl 3-oxovalerate were added dropwise to a solution of 14.2 g of 3,5,5-trimethylhexanal and 5 drops of piperidine in 100 ml of ethanol, and the mixture was stirred for two hours. The solvent was then distilled off, and the residue (25 g of a light-yellow oil) was used without further purification for the next step.

### Example 1b

### Methyl 5,7,7-trimethyl-2-propionyloctanoate

At 70° C. and under a hydrogen pressure of 10 bar, a solution of 25.0 g of the reaction product from example 1a was hydrogenated for 10 hours using 0.3 g of 10% palladium-on-carbon in 150 ml of ethanol. The catalyst was then filtered off and washed with ethanol, and the eluate was freed from the solvent. 24.7 g of the title compound remained as a light-yellow oil.

¹1 H-NMR (CDCl₃): δ=0.9 (s, 9H); 0.9 (d, 3H); 1.1 (t,3H); 1.0-1.3 (m, 4H); 1.5 (m, br., 1H); 1.8-1.9 (m, 2H); 2.45-2.6 (m, 2H); 3.4 (t, 1H); 3.7 (s, 3H).

### Example 1c

### 5-Ethyl-2-methyl-6-(3,5,5-trimethylhexyl)-[1,2,4]triazolo[1,5-a]pyrimidin-7-ol

A solution of 3.6 g of the keto ester from example 1b, 1.0 g of 3-amino-5-methyl-4H-1,2,4-triazole and 0.38 g of p-toluenesulfonic acid in mesitylene was stirred at 170° C. for 3.5 hours, during which time some solvent distilled off. The remaining mesitylene was then distilled off under reduced pressure, the residue was taken up in dichloromethane and washed with sat. NaHCO₃ solution, water and sat. NaCl solution and the solvent was removed. This gave 0.98 g of the title compound of m.p. 183-185° C.

### Example 1d

### 7-Chloro-5-ethyl-2-methyl-6-(3,5,5-trimethylhexyl)-[1,2,4]triazolo[1,5-a]pyrimidine

A solution of 0.98 g of the hydroxytriazolopyrimidine from example 1c in 10 ml of phosphorus oxychloride was heated under reflux for 3.5 hours. Excess phosphorus oxychloride was then distilled off and the residue was taken up in dichloromethane and stirred into water. After phase separation, the organic phase was washed with sat. NaHCO₃ solution, water and sat. NaCl solution and then dried, and the solvent was removed. This gave 0.95 g of the title compound as a yellow oil.

¹H-NMR (CDCl₃): δ=0.9 (s, 9H); 1.1 (d, 3H); 1.2 (dd, 1H); 1.3 (dd, 1H); 1.4 (t, 3H); 1.35-1.45 (m, 1H); 1.5-1.6 (m, 1H); 1.7 (m, br., 1H); 2.6 (s, 3H); 2.7-2.9 (m, 2H); 3.0 (q, 2H).

### Example 1e

### 5-Ethyl-2-methyl-6-(3,5,5-trimethylhexyl)-[1,2,4]triazolo[1,5-a]pyrimidin-7-ylamine

At 130° C., a solution of 1.0 g of the triazolopyrimidine from example 1d was stirred in an autoclave in 50 ml of 1,4-dioxane with 1.5 ml of (liquid) ammonia under autogenous pressure for 48 hours. After venting, the solvent was removed from the reaction mixture and the residue was taken up in a water/ethyl acetate mixture. After phase separation, the aqueous phase was extracted with ethyl acetate. The combined organic phases were washed with water and sat. NaCl solution and then dried, and the solvent was removed. The residue was digested with methyl tert-butyl ether (MtBE). 0.46 g of the title compound of m.p. 180-181° C. crystallized out.

TABLE I
Compounds of the formula I

| | | | | | Phys. |
|---|---|---|---|---|---|
| | | | | | Data |
| | | | | | (m.p. |
| No. | R¹ | R² | A | R³ | [° C.]) |
| I-1 | (CH₂)₂CH(CH₃)CH₂C(CH₃)₃ | CH₂CH₃ | N | CH₃ | 180-181 |
| I-2 | (CH₂)₇CH₃ | CH₂CH₃ | N | CH₃ | 184-185 |
| I-3 | (CH₂)₇CH₃ | CH₂CH₃ | CH | CH₃ | 149-150 |
| I-4 | (CH₂)₉CH₃ | CH₂CH₃ | CH | CH₃ | 130-131 |
| I-5 | (CH₂)₃—O—(CH₂)₈CH₃ | CH₂CH₃ | CH | CH₃ | 93-95 |
| I-6 | (CH₂)₃—O—(CH₂)₃CH₃ | CH₂CH₃ | CH | CH₃ | 131-133 |
| I-7 | (CH₂)₅CH₃ | CH₂CH₃ | CH | CH₃ | 154-156 |
| I-8 | (CH₂)₃—O—CH₂C(CH₃)₃ | CH₂CH₃ | CH | CH₃ | 124-126 |
| I-9 | CH₂CH(CH₃)CH₂CH₂CH₃ | CH₂CH₃ | CH | CH₃ | 173-174 |
| I-10 | CH₂CH(C₂H₅)(CH₂)₃CH₃ | CH₂CH₃ | CH | CH₃ | 139-140 |
| I-11 | CH₂CH(C₂H₅)₂ | CH₂CH₃ | CH | CH₃ | 146-147 |
| I-12 | (CH₂)₇CH₃ | CH₂CH₃ | N | CH₃ | 184-185 |
| I-13 | (CH₂)₅CH₃ | CH₂CH₃ | N | CH₃ | 187-188 |
| I-14 | (CH₂)₈CH₃ | CH₂CH₃ | N | CH₃ | 151-152 |

### Examples of the Action Against Harmful Fungi

The fungicidal action of the compounds of the formula I was demonstrated by the following experiments:

The active compounds were prepared as a stock solution comprising 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent/emulsifier of 99/1. The mixture was then made up to 100 ml with water. This stock solution was diluted with the solvent/emulsifier/water mixture described to the concentration of active compounds stated below.

Use example 1—Activity against peronospora of grapevines caused by Plasmopara viticola

Leaves of potted vines were sprayed to runoff point with an aqueous suspension comprising 250 ppm of active compound. The next day, the undersides of the leaves were inoculated with an aqueous sporangia suspension of Plasmopara viticola. The vines were then initially placed in a water vapor-saturated chamber at 24° C. for 48 hours and then in a greenhouse at temperatures between 20 and 30° C. for five days. After this time, the plants were once more placed in a humid chamber for 16 hours to accelerate the eruption of sporangiophores. The extent of the development of the infection on the undersides of the leaves was then determined visually.

In this test, the plants which had been treated with 250 ppm of the active compounds I-1, I-3 to I-5, I-9, I-10 or I-11 showed no or at most 5% infection, whereas the untreated plants were 100% infected.

Comparison with the closest prior art showed the following results:

| Comp. | From | | Infection |
|---|---|---|---|
| No. | document | Structure | in % |
| I-4 | according tothe invention | 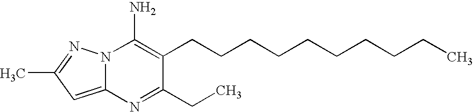 | 3 |
| 28 | EP-A 141317 | 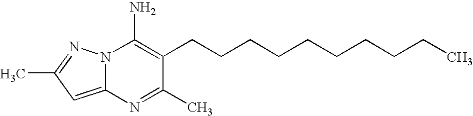 | 40 |

| | |
|---|---|
| untreated | 100 |

Use example 2—Activity against late blight on tomatoes caused by Phytophthora infestans, three day protective treatment

Leaves of potted tomato plants were sprayed to runoff point with an aqueous suspension having the concentration of active compound stated below. After three days, the leaves were infected with an aqueous sporangia suspension of Phytophthora infestans. The plants were then placed in a water vapor-saturated chamber at temperatures between 18 and 20° C. After 6 days, the late blight on the untreated, but infected control plants had developed to such an extent that the infection could be determined visually in %.

In this test, the plants which had been treated with 250 ppm of the active compound I-2 showed an infection of 3%, whereas the untreated plants were 90% infected.

1-13. (canceled)

14. An azolopyrimidine of formula I 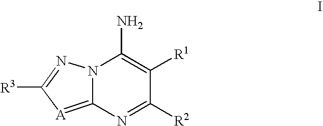
in which the substituents are as defined below:
R¹ is C₅-C₁₂-alkyl or C₅-C₁₄-alkoxyalkyl, where the aliphatic groups may be substituted by one to three of the following selected from the group consisting of:
cyano, nitro, hydroxyl, C₃-C₆-cycloalkyl, C₁-C₆-alkylthio, and NR^{a}R^{b};
R^{a}, R^{b} are hydrogen or C₁-C₆-alkyl;
R² is CHR^{x}CH₃, cyclopropyl, CH═CH₂ or CH₂CH═CH₂;
R^{x} is hydrogen, CH₃, CH₂CH₃ or halomethyl;
R³ is CH₃ and, if A is CH, additionally hydrogen.

15. The azolopyrimidine of formula I according to claim 14 in which R² is CH₂CH₃, CH═CH₂ or CH₂CH═CH₂.

16. The azolopyrimidine of formula I according to claim 14 in which R³ is methyl.

17. The azolopyrimidine of formula I according to claim 14 in which R¹ is an unsubstituted straight-chain or mono-, di-, or tribranched alkyl chain having up to 12 carbon atoms.

18. The azolopyrimidine of formula I according to claim 14 in which R² is ethyl.

19. The azolopyrimidine of formula I according to claim 14, selected from the group consisting of:
5-ethyl-2-methyl-6-(3,5,5-trimethylhexyl)-[1,2,4]triazolo[1,5-a]pyrimidin-7-ylamine;
5-ethyl-2-methyl-6-octyl-[1,2,4]triazolo[1,5-a]pyrimidin-7-ylamine;
5-ethyl-6-octylpyrazolo[1,5-a]pyrimidin-7-ylamine;
6-decyl-5-ethyl-2-methylpyrazolo[1,5-a]pyrimidin-7-ylamine;
5-ethyl-2-methyl-6-(3-nonyloxypropyl)pyrazolo[1,5-a]pyrimidin-7-ylamine;
6-(3-butoxypropyl)-5-ethyl-2-methylpyrazolo[1,5-a]pyrimidin-7-ylamine;
5-ethyl-6-hexyl-2-methylpyrazolo[1,5-a]pyrimidin-7-ylamine;
6-[3-(2,2-dimethylpropoxy)propyl]-5-ethyl-2-methylpyrazolo[1,5-a]pyrimidin-7-ylamine;
5-ethyl-2-methyl-6-(2-methylpentyl)pyrazolo[1,5-a]pyrimidin-7-ylamine;
5-ethyl-6-(2-ethylheptyl)-2-methylpyrazolo[1,5-a]pyrimidin-7-ylamine;
5-ethyl-6-(2-ethylbutyl)-2-methylpyrazolo[1,5-a]pyrimidin-7-ylamine;
5-ethyl-2-methyl-6-octyl[1,2,4]triazolo[1,5-a]pyrimidin-7-ylamine;
5-ethyl-2-methyl-6-pentyl[1,2,4]triazolo[1,5-a]pyrimidin-7-ylamine; and
5-ethyl-2-methyl-6-nonyl[1,2,4]triazolo[1,5-a]pyrimidin-7-ylamine.

20. A process for preparing an azolopyrimidine of formula I 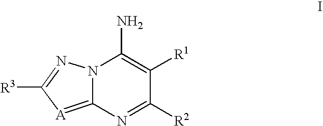
in which the substituents are as defined below:
R¹ is C₅-C₁₂-alkyl or C₅-C₁₄-alkoxyalkyl, where the aliphatic groups may be substituted by one to three of the following selected from the group consisting of:
cyano, nitro, hydroxyl, C₃-C₆-cycloalkyl, C₁-C₆-alkylthio, and NR^{a}R^{b};
R^{a}, R^{b} are hydrogen or C₁-C₆-alkyl;
R² is CHR^{x}CH₃, cyclopropyl, CH═CH₂ or CH₂CH═CH₂;
R^{x} is hydrogen, CH₃, CH₂CH₃ or halomethyl;
R³ is CH₃ and, if A is CH, additionally hydrogen;
wherein β-keto esters of the formula II 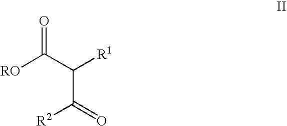
in which R is C₁-C₄-alkyl are reacted with an aminoazole of the formula III 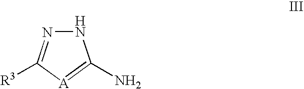
in which R³ is as defined above, to give 7-hydroxyazolopyrimidines of the formula IV 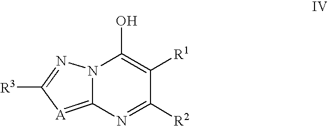
which are halogenated to give compounds of the formula V 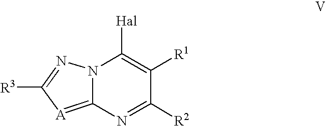
in which Hal is chlorine or bromine and V is reacted with ammonia.

21. The compounds selected from the group consisting of formulae IV and V: 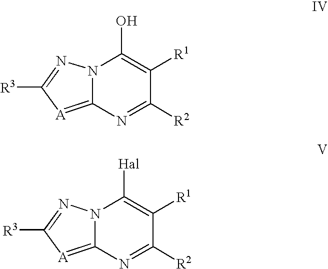
wherein
R¹ is C₅-C₁₂-alkyl or C₅-C₁₄-alkoxyalkyl, where the aliphatic groups may be substituted by one to three of the following selected from the group consisting of:
cyano, nitro, hydroxyl, C₃-C₆-cycloalkyl, C₁-C₆-alkylthio, and NR^{a}R^{b};
R^{a}, R^{b} are hydrogen or C₁-C₆-alkyl;
R² is CHR^{x}CH₃, cyclopropyl, CH═CH₂ or CH₂CH═CH₂;
R^{x} is hydrogen, CH₃, CH₂CH₃ or halomethyl;
R³ is CH₃ and, if A is CH, additionally hydrogen.

22. A process for preparing an azolopyrimidine of the formula I 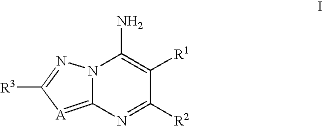
in which the substituents are as defined below:
R¹ is C₅-C₁₂-alkyl or C₅-C₁₄-alkoxyalkyl, where the aliphatic groups may be substituted by one to three of the following selected from the group consisting of:
cyano, nitro, hydroxyl, C₃-C₆-cycloalkyl, C₁-C₆-alkylthio, and NR^{a}R^{b};
R^{a}, R^{b} are hydrogen or C₁-C₆-alkyl;
R² is CHR^{x}CH₃, cyclopropyl, CH═CH₂ or CH₂CH═CH₂;
R^{x} is hydrogen, CH₃, CH₂CH₃ or halomethyl;
R³ is CH₃ and, if A is CH, additionally hydrogen;
wherein acylcyanides of the formula VI 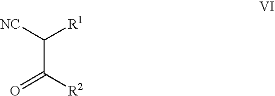
are reacted with an aminoazole of formula III

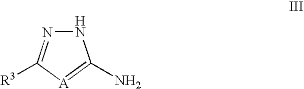

23. A composition comprising a solid or liquid carrier and an azolopyrimidine of formula I 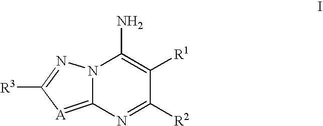
in which the substituents are as defined below:
R¹ is C₅-C₁₂-alkyl or C₅-C₁₄-alkoxyalkyl, where the aliphatic groups may be substituted by one to three of the following selected from the group consisting of:
cyano, nitro, hydroxyl, C₃-C₆-cycloalkyl, C₁-C₆-alkylthio, and NR^{a}R^{b};
R^{a}, R^{b} are hydrogen or C₁-C₆-alkyl;
R² is CHR^{x}CH₃, cyclopropyl, CH═CH₂ or CH₂CH═CH₂;
R^{x} is hydrogen, CH₃, CH₂CH₃ or halomethyl;
R³ is CH₃ and, if A is CH, additionally hydrogen.

24. The composition according to claim 23, comprising a further active compound.

25. Seed comprising an azolopyrimidine of formula I 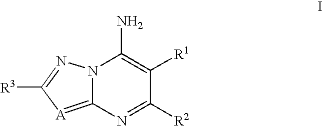
in which the substituents are as defined below:
R¹ is C₅-C₁₂-alkyl or C₅-C₁₄-alkoxyalkyl, where the aliphatic groups may be substituted by one to three of the following selected from the group consisting of:
cyano, nitro, hydroxyl, C₃-C₆-cycloalkyl, C₁-C₆-alkylthio, and NR^{a}R^{b};
R^{a}, R^{b} are hydrogen or C₁-C₆-alkyl;
R² is CHR^{x}CH₃, cyclopropyl, CH═CH₂ or CH₂CH═CH₂;
R^{x} is hydrogen, CH₃, CH₂CH₃ or halomethyl;
R³ is CH₃ and, if A is CH, additionally hydrogen;
in amounts of 1 to 1000 g per 100 kg of seed.

26. A method for controlling phytopathogenic harmful fungi, wherein the fungi or the materials, plants, the soil or seed to be protected against fungal attack are treated with an effective amount of an azolopyrimidine of formula I 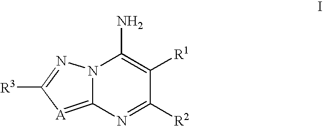
in which the substituents are as defined below:
R¹ is C₅-C₁₂-alkyl or C₅-C₁₄-alkoxyalkyl, where the aliphatic groups may be substituted by one to three of the following selected from the group consisting of:
cyano, nitro, hydroxyl, C₃-C₆-cycloalkyl, C₁-C₆-alkylthio, and NR^{a}R^{b};
R^{a}, R^{b} are hydrogen or C₁-C₆-alkyl;
R² is CHR^{x}CH₃, cyclopropyl, CH═CH₂ or CH₂CH═CH₂;
R^{x} is hydrogen, CH₃, CH₂CH₃ or halomethyl;
R³ is CH₃ and, if A is CH, additionally hydrogen.
